(19) United States
(12) Patent Application Publication
(10) Pub. No.: US 2018/0000736 A1
(43) Pub. Date: Jan. 4, 2018

(54) METHODS FOR THE REGENERATION OF ARTICULAR CARTILAGE IN VIVO

(71) Applicant: University of Iowa Research Foundation, Iowa City, IA (US)

(72) Inventors: James A. Martin, Iowa City, IA (US); Yin Yu, Iowa City, IA (US); Dong Rim Seol, Iowa City, IA (US); Aliasger K Salem, Coralville, IA (US); Behnoush Khorsand Sourkohi, Iowa City, IA (US); Anh-Vu T Do, Iowa City, IA (US); Marc Brouillette, Coralville, IA (US)

(21) Appl. No.: 15/541,737

(22) PCT Filed: Jan. 7, 2016
(86) PCT No.: PCT/US2016/012456

§ 371 Date: Jul. 6, 2017

Related U.S. Application Data
(60) Provisional application No. 62/101,174, filed on Jan. 8, 2015.

(51) Int. Cl. A61K 9/14; A61K 38/30; A61K 38/18

(52) U.S. Cl. CPC A61K 9/146; A61K 38/1841; A61K 38/30

(57) ABSTRACT

A pharmaceutical composition is provided that is useful to enhance the repair of articular cartilage, to treat a joint injury or to prevent, inhibit or treat osteoarthritis in a mammal. The composition may include an effective amount of an isolated protein that is a chemoattractant for chondrogenic progenitor cells and/or an effective amount of an isolated protein that is a chondro genic factor or a nucleic acid sequence that encodes a chondrogenic factor.

### CROSS-REFERENCE TO RELATED APPLICATIONS

This application claims the benefit of the filing date of U.S. application Ser. No. 62/101,174, filed on Jan. 8, 2015, the disclosure of which is incorporated by reference herein.

### BACKGROUND

Adult rheumatic diseases occur in many forms. One common rheumatic disease is arthritis, of which there are many types. Common symptoms of arthritis include: swelling in one or more joints, stiffness around the joints that lasts for at least 1 hour in the early morning, constant or recurring pain or tenderness in a joint, difficulty using or moving a joint normally, and warmth and redness in a joint.

The most common type of arthritis is osteoarthritis, This type of arthritis affects an estimated 21 million adults in the United States, Osteoarthritis primarily affects cartilage. Cartilage is composed of specialized cells called chondrocytes that produce a large amount of extracellular matrix composed of collagen fibers, substances rich in proteoglycan and elastin fibers, Cartilage is classified in three types: elastic cartilage, hyaline cartilage and fibrocartilage Compared to other connective tissues, cartilage grows and repairs more slowly. Hyaline cartilage is the most difficult to repair. Damaged hyaline cartilage is usually replaced by fibrocartilage scar tissue, which is not as durable. In osteoarthritis, the cartilage begins to fray and may entirely wear away. Disability results most often when the disease affects the spine and the weight-bearing joints (the knees and hips).

The pain, immobility, and general disability associated with osteoarthritis are familiar to most people who reach old age. Fortunately, artificial joint replacements have made it possible to permanently restore normal joint function in older patients. However, some less common forms of osteoarthritis, like post-traumatic osteoarthritis (PTOA), preferentially affect people who are too young for joint replacement. Because there are no viable alternatives to joint replacement, patients with PTOA often suffer disability and morbidity comparable to patients with chronic heart disease. In addition, focal damage to cartilage caused by common forms of joint injury like ACL rupture seldom heals spontaneously and may initiate PTOA.

A number of methods to enhance cartilage healing involve grafting chondrocytes or stern cells. Stern cell-based tissue engineering treatments using bone marrow rnesenchymal stern cells (BMSCs) (Pittenger et al,, 1999), as well as adipose stern cells (ASCs) (Erickson et al., 2012), for adult human articular cartilage repair have drawn great attention and been extensively studied (Tuan, 2006). Although substantial success has been achieved, the low yields of BMSCs, and phenotypic alteration during prolonged in vitro cultivation often limited their application in clinics. Moreover, chondrogenic activity of BMSCs is age- and OA-dependent, and ASCs generate repair tissue with mechanical properties that are inferior to hyaline cartilage. In addition, pluripotent progenitor cells from multiple joint tissues including synovium (De Bari et al., 2001), infrapatellar fat pad (Wickham et al., 2003), and meniscus (Shen et al., 2014), have recently been shown to have articular cartilage repair potential in short-term studies. However, current strategies often fail to regenerate permanent hyaline cartilage that is well integrated with the surrounding matrix and biologically and mechanically similar to native cartilage. For example, stern cells may also display a hypertrophic phenotype upon chondrogenic induction, which is undesirable for restoring an articular surface (Johnstone et al., 2013). Risks and crucial barriers to stem cell therapy, like pathogen transmission and tumorigenesis, and complex ethical and regulatory issues, have limited clinical implementation (Fodor, 2003; Prockop, 2009).

### SUMMARY

The present invention exploits the intrinsic repair powers of chondrogenic progenitor cells (CPCs) residing in cartilage and exogenously delivered agents that dramatically enhance the repair capacity of CPCs. In one embodiment, the invention provides a composition having a chemoattractant and optionally a chondrogenic protein. In one embodiment, the invention provides a composition having an amount of a chemoattractant effective to draws local CPCs to any site on the cartilage surface where the compositions is placed (e.g., in damaged cartilage). In one embodiment, the invention provides an injectable composition with a chemoattractant that draws local CPCs to any site on the cartilage surface where the composition is placed. In one embodiment, the invention provides a composition having hydrogel loaded with a chemoattractant that draws local CPCs to any site on the cartilage surface where the gel is placed. In one embodiment, the invention provides an injectable composition having hydrogel loaded with a chemoattractant that draws local CPCs to any site on the cartilage surface where the gel is placed. The composition may also contain a chondrogenic protein, e.g., one encapsulated in a delayed-release formulation that drives the production of hyaline cartilage matrix by CPCs after they have migrated into the composition. The combination of materials and sequential (multi-phase) delivery of chemotactic and chondrogenic factors offers a unique practical advantage in treating cartilage lesions, for instance, in a single arthroscopic procedure. In one embodiment, the composition does not include cells, e.g., autologous or allogenic cells including MSCs or ASCs.

As described herein, it was determined whether SDF-1α, a potent CPC chemoattractant, would improve the quality of cartilage regeneration using the potential of a migratory CPC population that responded chemotactically to cell death and rapidly repopulates the injured cartilage matrix. It was hypothesized that increased recruitment of CPCs by rhSDF-1α would promote the formation of cartilage matrix upon chondrogenic induction. Full-thickness bovine chondral defects were filled with hydrogel comprised of fibrin and hyaluronic acid and containing rhSDF-1α. Cell migration was monitored, followed by chondrogenic induction. Regenerated tissue was evaluated by histology, immunohistochemistry, and scanning electron microscopy, Push-out tests as well as unconfined compression test were performed to assess the strength of tissue integration and the mechanical properties of regenerated cartilage. rhSDF-1α dramatically improved CPCs recruitment to defects at 12 days. After 6 weeks chondrogenesis, repair tissue cell morphology, proteoglycan density, and ultrastructure, were similar to native cartilage. Neocartilage generated in rhSDF-1α-containing defects showed significantly greater interfacial strength than controls, and acquired mechanical properties comparable to native cartilage tissues.

Thus, stimulating local CPCs recruitment prior to treatment with chondrogenic factors significantly improves the mechanical properties of tissues formed in chondral defects. This approach may be implemented in vivo as a one-step procedure by staging the sequential release of chemokine and chondrogenic factors, e.g., from within the hydrogel, to regenerate healthy hyaline cartilage. This lowers risk and morbidities associated with other more invasive approaches that require multiple surgical procedures and/or cell harvests. Moreover, the present cartilage repair/healing strategy may generate cartilage of a quality that is superior to other methods.

In one embodiment, administration of an effective amount of a composition of the invention allows for enhanced repair of cartilage, e.g., in the joints between bones, the rib cage, the ear, the nose, the bronchial tubes and/or the intervertebral discs, relative to cartilage that is not treated with the composition. In one embodiment, the invention includes an injectable composite hydrogel, for instance, formed of hyaluronic acid (HA), fibrin or a combination thereof, loaded with a chemoattractant such as stromal derived factor 1 alpha (SDF-1alpha), an alarmin, e.g., HMGB1, or IL-8, in an amount that draws CPCs to the cartilage surface, and optionally having a chondrogenic protein (for instance, a member of the TGF-beta superfamily, e.g., a TGFbeta or a BMP including but not limited to one of TGF-β1, -β2 or -β3, one of BMP2, BMP4 or BMP7, or IGF-1, or any combination thereof) in an amount to enhance production of hyaline cartilage matrix by CPCs after they have migrated into the gel. In one embodiment, compositions and methods of using these compositions to promote repair of articular cartilage damage and restoration of normal hyaline cartilage, employ hydrogels comprising rhSDF-1α, fibrin and hyaluronic acid.

In one embodiment, the chemoattractant protein has at least 80%, 82%, 85%, 87%, 90%, 92%, 95%, 97%, 98%, or 99% or more amino acid sequence identity to one of SEQ ID NO: 1, 2 or 12. In one embodiment, the chrondrogenic factor comprises a member of the TGF-beta superfamily, e.g., a TGFbeta or a BMP. In one embodiment, the chondrogenic protein has at least 80%, 82%, 85%, 87%, 90%, 92%, 95%, 97%, 98%, or 99% or more amino acid sequence identity to one of SEQ ID NO: 3-11. The polypeptides in the composition of the invention include those with conservative substitutions, e.g., relative to the polypeptide having SEQ ID NO:1, SEQ ID NO:2, SEQ ID NO:3,SEQ ID NO:4, SEQ ID NO:5, SEQ ID NO:6, SEQ ID NO:7, SEQ ID NO:8, SEQ ID NO:9, SEQ ID NO:10, or SEQ ID NO:11. In one embodiment, the polypeptide has 1, 2, 5 or up to 20 (or any integer in between) amino acid substitutions. Non-conservative substitutions, as well as combinations of conservative and non-conservative substitutions, are also envisioned.

Further provided is a composition comprising isolated microparticles or isolated nanoparticles having the chonclrogenic protein in a hydrogel comprising the chemoattractant. In one embodiment, the hydrogel and microparticles or nanoparticles are formed of different materials or different ratios of materials to provide for different release profiles. For example, a hydrogel having the chemoattractant releases the chemoattractant within minutes, or up to or over hours or 1, 2, 3, 4, 7, 14 or 21, or more, clays after administration while microparticles having the chondrogenic factor release that factor over 1, 2, 3, 4, 6, or 7 or more weeks (“sustained” or “delayed” release) after administration.

Also provided is a method to enhance the repair of articular cartilage, to restore hyaline cartilage and/or inhibit cartilage damage in a mammal. The method includes administering to the mammal a composition comprising an effective amount of a chemoattractant and optionally a chondrogenic protein. In one embodiment, the amount enhances the amount or level of normal hyaline cartilage. In one embodiment, the composition is injected. In one embodiment, the composition does not include cells.

Further provided is a method to prevent, inhibit or treat osteoarthritis or PTOA in a mammal. The method includes comprising administering to the mammal a composition comprising an effective amount of a chemoattractant and optionally a chondrogenic protein. In one embodiment, the composition is injected. In one embodiment, the composition comprises a hydrogel. In one embodiment, the composition comprises nanoparticles or microparticles. In one embodiment, the microparticle comprises polylactic acid or copolymers thereof, e.g., polylactic co-glycolic acid) (PLGA) copolymers. In one embodiment, the microparticles comprise a TGFbeta superfamily member, for example, at least one of TGF-β1, -β2 or -β3 or at least one of BMP2, BMP4 or BMP7, or IGF-1, or a combination thereof. In one embodiment, the composition is injected. In one embodiment, the composition is a multi-phase release formulation. In one embodiment, the composition comprises a hydrogel having one or more chemoattractants and microparticles having one or more chondrogenic factors. In one embodiment, the hydrogel and microparticles are formed of different materials or different ratios of materials. In one embodiment, the different materials or different ratios of materials provide for different release profiles.

Further provided is the composition as described herein for use in medical therapy, e.g., to enhance the repair of articular cartilage, to treat a joint injury or to prevent, inhibit or treat osteoarthritis

### BRIEF DESCRIPTION OF THE FIGURES

FIG. 1, Fabrication and characterization of IPN hydrogel. (A) A schematic presentation of IPN hydrogel fabrication, fibrin hydrogel and HA polymer were blended and cross-linked to form interpenetrating polymer network; Macroscopic view of IPN scaffold (B) showed white color and SEM images showed interpenetrated polymer fibers (C) and interconnected pores (D, arrow heads). rhSDF-1α loaded IPN scaffold maintained its integrity in PBS during drug release study for 14 days (E-G); rhSDF-1α protein continued to released from IPN over 14 days (H). Data was present as mean ±SD (n=4 for each time point). Scale bar, B: 5 mm, E-G: 4 mm. Encapsulated CPCs were largely viable (green fluorescence) at day 1 (L), 7 (J), (21), with minimal number of dead cells presented (red fluorescence). Average cell viability maintained over 90% for different time points (L). (n=6 for each time point) Scale bar, B: 5 mm, E-G: 4 mm, and L-K: 500 μm. 75×68 mm (300×300 DPI).

FIG. 2. SDF-1α expression and in vitro cell migration. (A) Monolayer cultured CPCs were positively stained (red fluorescence) for SDF-1α and CXCR4, while NCs were largely negative for bot markers with only DAPI staining (blue fluorescence); Positive SDF-1α staining were present in impacted cartilage tissue sections, while not in those from healthy un-impacted cartilage; RT-PCT showed profound up-regulation of SDF-1α (>13-fold) and CXCR4 (>3.5 fold) for CPCs in comparison with NCs. (B) Schematic representation of experimental design. Stacked confocal images from different time points showed that rhSDF-1α initiated dramatic cell migration in comparison with PBS control in a concentration and time dependent manner. (C) Quantification of high magnitude images (Day 12H) confirmed significantly higher (P<0.0001, n=8) number of progenitor cells migrated in response to rhSDF-1α, and DNA quantification also suggested much higher (P=0.0227, n=8) dsDNA content in rhSDF-1α loaded IPN compared with controls. Scale bar, A: 200 μm and C: 500 μm. (*) indicates significant difference (P<0.05). 71×61 mm (300×300 DPI).

FIG. 3. Histological and quantitative analysis for cartilage tissue regeneration. (A-L) Safranin-O/fast green staining of regenerated cartilage tissue sections. Stronger Safranin-O positive staining and more organized proteoglycan deposition presented in rhSDF-1α treated group both at 3W (D-F) and 6W (J-L). At 3W, cells displayed unique spindle shape, characteristic of CPCs in both groups (C&F), while differentiated into cobblestone-like morphology at 6W (I&L). HT indicates host tissue, and RT indicates regenerated tissue. Quantitative analysis of sGAG, water content per normalized to wet weight, and cell density per filed (n=8 for each group). Scale bar, A, D, G, J: 1 mm; B, E, H, K: 200 μm; C, F, I, L: 50 μm. (*) indicates significant difference (P<0.05). 95×108 mm (300×300 DPI).

FIG. 4. Immunohistochemical examination for articular cartilage specific proteins. Type II collagen (A-B) and aggrecan (C-D) immunohistochemical staining. Significant staining for rhSDF-1α treated group (B&D) in comparison with IPN only groups with the absence of rhSDF-1α (A&C); zonally organized lubricin staining (F) in SDF (+) groups, while not in SDF (−) groups (E); SDF (+) groups showed continuous staining for all three proteins between host cartilage and regenerated cartilage tissue, especially at the superficial zone (B, D & F, insets), but not in SDF (+) groups. All negative control without primary antibodies only lightly stained for background (A, D & G). Scale bar, 200 μm and 1 mm (insets). 70×58 mm (300×300 DPI).

FIG. 5. Assessment of cartilage tissue integration. (A) Complete repair of cartilage defect for SDF (+) groups (lower left) but not for SDF (−) groups (upper left), macroscopically; Safranin-O staining showed continuous proteoglycan rich matrix projected from repair tissue to host cartilage tissue with seamless connection in SDF (+) groups (lower middle), while mildly stained repair tissue loosely connected with native cartilage for SDF (−) groups (upper middle); In SDF (+) groups, type II collagen, showing well-organized strong intensity staining in the entire matrix of the interfacial area (lower right), while in SDF (−) groups (upper right), staining only presented partially at the tissue interface; (B) Apparatus and scheme (dashed inset) for push-out test; (C) Both peak force (p=0.0004) and stress (p<0.0001) were significantly higher (>20 fold) in SDF (+) (n=9) than SDF (−) (n=6) groups, (D) SEM images showed continuous cells ingrowth from the surface (I) and cross-section at the tissue interface (III), also interconnected extra cellular matrix (II) with entangled collagen fibers (IV). (*) indicates significant difference (P<0.05). 67×38 mm (300×300 DPI).

FIG. 6. Biomechanical characterization of regenerated cartilage tissue. (A) SEM images showing morphology of cells and pattern of ECM fibers of host cartilage and regenerated cartilage tissue; (B) similar sGAG content and water content of regenerated cartilage as host cartilage, while differ significantly from empty IPN gel; (C) Apparatus and scheme (dashed inset) for stress-relaxation test, and gross appearance of three different cartilage tissue under test; (D) stress-strain curve for three kinds of tested cartilage tissue under 1 mm/s (upper) and 2 mm/s (lower) loading rate, respectively; (E) maximum force, maximum stress, equilibrium stress and Young's modulus for tibial plateau cartilage (TPC), regenerated cartilage (REGC), and femoral condyle cartilage (FCC) under 1 mm/s and 2 mm/s loading rate. Data presented are mean±SD for 8-9 different samples for each group. (*) indicates significant differences (P<0.05). 78×60 mm (300×300 DPI).

FIG. 7. Exemplary sequences for SDF1-alpha (SEQ ID NO:1), HMGB1 (SEQ ID NO:2), IL8 (SEQ ID NO:12), human TGFα3 (SEQ ID NOs: 3 and 4), human BMP2 (SEQ ID NOs: 5 and 6), human BMP4 (SEQ ID NO:7), human BMP7 (SEQ ID NO:8), human insulin-like growth factor 1 (SEQ ID NO:9), human TGF-beta1 (SEQ ID NO:10), or human TGF-beta2 (SEQ ID NO:11).

### DETAILED DESCRIPTION

A series of studies have identified sub-populations of stem/progenitor cells in articular cartilage as well as from repair tissue of late-stage osteoarthritis (Dowthwaite et al., 2004; Alsalameh et al., 2004; Koelling et al., 2009). These cells, often referred as chondrogenic progenitor cells (CPCs), respond to various chemokines and cytokines and migrate towards damaged cartilage tissue (Seol et al., 2014). They also exhibit other characteristics of stem/progenitor cells including an apparent potential for repairing cartilage defects (Koelling et al., 2009; Seol et al., 2012; Seol et al,, 2014). CPCs are thought to be candidates for regenerative therapy of osteoarthritis (Dealy, 2012), yet no studies have reported articular cartilage regeneration using CPCs.

To repair full thickness articular cartilage defects in a bovine osteochondral explant model, the recruitment of migratory progenitor cells to IPN was enhanced using recombinant human SDF-1α alpha (rhSDF-1α) to recruit CPCs followed by treatments to initiate chondrogenic differentiation. These sequential manipulations likely result in near complete restoration of cartilage matrix within the defect and improved integration with host tissue compared with controls lacking one or both factors.

In one embodiment, the present cartilage repair strategy may be employed to treat patients at risk for PTOA because they have damaged their articular cartilage. As of 2006 approximately 12% of the overall prevalence of symptomatic OA was attributable to PTOA of the hip, knee, or ankle. This corresponds to approximately 5.6 million individuals in the United States being affected by PTOA. The present treatment is designed to prevent PTOA, which may substantially lower burden, and may be more cost-effective and present fewer risks to patients than alternatives that are currently in use.

### Definitions

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo. Illustrative vectors include, for example, plasmids, viral vectors, liposomes, nanoparticles, or microparticles and other delivery vehicles. In one embodiment, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell, Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by heterologous hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

Gene transfer refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers, A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e,, on a nucleotide-by-nucleotide basis) over the window of comparison, The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As used herein, “substantially pure” or “purified” means an object species is the predominant species present (i.e., on a molar basis it is more abundant than any other individual species in the composition), for instance, a substantially purified fraction is a composition wherein the object species comprises at least about 50 percent (on a molar basis) of all macromolecular species present. Generally, a substantially pure composition will comprise more than about 80 percent of all macromolecular species present in the composition, or more than about 85%, about 90%, about 95%, and about 99%. The object species may be purified to essential homogeneity (contaminant species cannot be detected in the composition by conventional detection methods) wherein the composition consists essentially of a single macromolecular species.

### Preparation of Expression Cassettes

To prepare expression cassettes encoding a protein such as a CPC chemoattractant protein or a chondrogenic protein, or a peptide thereof, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotrophic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR in the practice of the invention.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, pure, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coil, the beta-glucuronidase gene (gus) of the uidA locus of E. coil, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e,g., by immunological means (ELISAs and Western blots) or by other molecular assays.

To detect and quantitate RNA produced from introduced recombinant DNA segments, RI-FOR may be employed. In this application of FOR, it is first necessary to reverse transcribe RNA into DNA, using enzymes such as reverse transcriptase, and then through the use of conventional FOR techniques amplify the DNA, In most instances FOR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and FOR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

### Polypeptides (Proteins) and Peptides

The isolated peptide or polypeptide of the invention can be synthesized in vitro, e.g., by the solid phase peptide synthetic method or by recombinant DNA approaches (see above). The solid phase peptide synthetic method is an established and widely used method. These peptides or polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, chemically modified derivatives of a given peptide or polypeptide, can be readily prepared. For example, amides of the peptide or polypeptide of the present invention may also be prepared by techniques well known in the art for converting a carboxylic acid group or precursor, to an amide. One method for amide formation at the C-terminal carboxyl group is to cleave the peptide or fusion thereof from a solid support with an appropriate amine, or to cleave in the presence of an alcohol, yielding an ester, followed by aminolysis with the desired amine.

Salts of carboxyl groups of a peptide or polypeptide may be prepared in the usual manner by contacting the peptide or polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

Other modifications of the peptide or polypeptide may be prepared by utilizing an N-acyl protected amino acid for the final condensation, or by acylating a protected or unprotected peptide or polypeptide. O-acyl derivatives may be prepared, for example, by acylation of a free hydroxy peptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In one embodiment, a peptide or polypeptide has substantial identity, e.g., at least 80% or more, e.g., 85%, 87%, 90%, 92%, 95%, 97%, 98%, 99% and up to 100%, amino acid sequence identity to one of SEQ ID NOs. 1-12, and may, when administered alone or in combinations, promote cartilage growth or repair.

Substitutions may include substitutions which utilize the D rather than L form, as well as other well known amino acid analogs, e.g., unnatural amino acids such as α,α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, a-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine.

Conservative amino acid substitutions may be employed—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide or polypeptide can readily be determined by assaying the specific activity of the peptide or polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic; trp, tyr, phe.

The invention also envisions a peptide or polypeptide with non-conservative substitutions. Non-conservative substitutions entail exchanging a member of one of the classes described above for another.

Acid addition salts of the peptide or polypeptide or of amino residues of the peptide or polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Formulations and Dosages

The polypeptides or peptides can be formulated as pharmaceutical compositions and administered to a mammalian host, such as a human patient in a variety of forms adapted to the chosen route of administration, e.g., orally or parenterally, by intravenous, intramuscular, topical, local, or subcutaneous routes. In one embodiment, the composition having isolated polypeptide or peptide is administered to a site of cartilage damage or suspected cartilage damage, or is administered prophylactically.

In one embodiment, the polypeptides or peptides may be administered by infusion or injection. Solutions of the polypeptides or peptides, or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, it may be preferable to include isotonic agents, for example, sugars, buffers or sodium chloride. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the polypeptides or peptides can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the polypeptides or peptides in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the polypeptides or peptides for use alone or with other agents will vary with the route of administration, the nature of the condition being treated and the age and condition of the patient and will be ultimately at the discretion of the attendant physician or clinician.

The polypeptides or peptides may be conveniently administered in unit dosage form; for example, containing 5 to 1000 mg, conveniently 10 to 750 mg, or conveniently 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

### Exemplary Delivery Vehicles

Delivery vehicles for the peptides or polypeptides in the compositions of the invention include, for example, naturally occurring polymers, microparticles, nanoparticles, and other macromolecular complexes capable of mediating delivery of a protein to a host. Vehicles can also comprise other components or functionalities that further modulate, or that otherwise provide beneficial properties. Such other components include, for example, components that influence binding or targeting to cells or physiological components, e.g., cartilage.

In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer comprising the chemoattractant protein and optionally the chondrogenic protein. In one embodiment, the composition is a hydrogel comprising the chemoattractant protein and optionally the chondrogenic protein. In one embodiment, the chemoattractant protein and/or the chondrogenic protein are in nanoparticles or microparticles. For example, the chondrogenic protein may be in nanoparticles or microparticles in a naturally occurring polymer that has the chemoattractant protein. Table provides exemplary materials for delivery vehicles that are formed of naturally occurring polymers and materials for particles.

TABLE 1

| Particle class | Materials |
|---|---|
| Natural materials or | Chitosan |
| derivatives | Dextran |
| | Gelatine |
| | Albumin |
| | Alginates |
| | Liposomes |
| | Starch |
| Polymer carriers | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials for particle formation include but are not limited to agar acrylic polymers, polyacrylic acid, poly acryl methacrylate, gelatin, poly(lactic acid), pectin(poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrrollidone, and shellac. Soluble starch and its derivatives for particle preparation include amylodextrin, amylopectin and carboxy methyl starch.

In one embodiment, the polymers in the nanoparticles or microparticles are biodegradable. Examples of biodegradable polymers useful in particles preparation include synthetic polymers, e.g., polyesters, poly(ortho esters), polyanhydricles, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a hydrogel. Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and include proteins like fibrin, collagen and gelatin, and polysaccharides like starch, alginate and agarose.

### Exemplary Compositions

SIF-1 has two isoforms (SDF-1α and SDF-1β), which are generated from the same gene by differential RNA splicing and, only differ by their C-terminus. In one embodiment, the chemoattractant protein in a composition of the invention comprises SDF-1α, also known as CXCL12, which is a member of the CXC chemokine family. SDF-1α is a key cytokine regulating stem cell migration and homing to sites of tissue damage, where they participate in tissue or organ regeneration. SDF-1α exerts its effects through binding to the cell surface receptor, CXCR4 (Hattori et al., 2001; Lagasse et al., 2000), which is expressed on many cell types. Seol and colleagues reported that SDF-1α and CXCR4 expression was highly upregulated in a migratory progenitor cell population found on articular cartilage surfaces within a few days after focal impact (Seol et al., 2012), and progenitor cells responded rigorously to SCF-1α, which suggests that SDF-1α plays a role in in situ articular cartilage repair by recruiting endogenous stern or progenitor cells. However, other CPC chemoattractant proteins may be employed in the composition, optionally in combination with SDF-1α.

In one embodiment, the delivery vehicle for the chemoattractant protein comprises fibrin and hyaluronic acid. Fibrin (also known as Factor 1a) is a fibrous, non-globular protein involved in the clotting of blood. It is formed by the action of the protease thrombin on fibrinogen which causes the latter to polymerize. When the lining of a blood vessel is broken, platelets are attracted forming a platelet plug. Fibrin forms long strands of tough insoluble protein that are laid down and are bound to the platelets.

Hyaluronic acid (also called hyaluronate or HA) is an anionic, nonsulfated glycosaminoglycan distributed widely throughout connective, epithelial, and neural tissues. HA is an important component of articular cartilage, where it is present as a coat around each cell (chondrocyte). When aggrecan monomers bind to hyaluronan in the presence of link protein, large, highly negatively charged aggregates form. These aggregates imbibe water and are responsible for the resilience of cartilage (its resistance to compression). The molecular weight (size) of hyaluronan in cartilage decreases with age, but the amount increases.

The unique biocompatibility and highly hydrated structure of fibrin/HA can mimic natural tissues and deliver biochemical cues (Klein et al., 2009; Slaughter et al., 2009). A composite interpenetrating hydrogel network (IPN) composed of fibrin and HA has been shown to exhibit mechanical properties that are far superior to either polymer alone. The excellent cell affinity of fibrin and delayed degradation of HA results in mutually beneficial effects on cartilage ECM synthesis (Rarnpichova et al., 2010).

In one embodiment, the composition further comprises a chondrogenic protein or a combination of chondrogenic proteins, which optionally are in microparticles to allow for differential release relative to the chemoattractant protein.

The invention will be further described by the following non-limiting example.

### EXAMPLE

### Materials and Methods

### IPN Hydrate Fabrication, Drug Release and Biocompatibility

IPN hydrogel consisted of HA-thrombin (Solution A) and fibrinogen (Solution B). For Solution A, 10 mg/mL hyaluronate (GelOne™, Zimmer Inc., Warsaw, Ind.) was mixed with same volume of 40 U/mL thrombin (TISSEEL™, Baxter Healthcare Corp., Westlake Village, Calif.). Solution B was 25 mg/mL fibrinogen (TISSEEL™, Baxter Healthcare Corp.) in Dulbecco's phosphate-buffered saline (DPBS, pH 7.4) with or without 400 ng/mL (or 200 ng/mL) rhSDF-1α (R&D Systems Inc., Minneapolis, Minn., USA). To form IPN, solution A and B were gently mixed together at a ratio of 1:1 at 4° C. The final concentrations of hyaluronate, thrombin, fibrinogen, and rhSDF-1α were 2.5 mg/L, 10 U/mL, 12.5 mg/mL, and 200 ng/mL respectively.

Cylindrical shaped IPN hydrogel disks (thickness of 2 mm and diameter of 4 mm) were fabricated in a plastic mold and kept in DPBS for future use. Protein release kinetics of rhSDF-1α were determined according to Sukegawa et al, (2012). Briefly, each IPN hydrogel disk was placed in a 24-well plate with 400 μL of DPBS per well and cultured at 37° C. Supernatants were collected at each time point (day 2, 4, 6, 8, 10, 12, and 14). 400 μL DPBS was added to replenish each well and samples were placed back for cultivation until next time point. Enzyme-linked immunosorbent assay (ELISA) was used for quantification according to the manufacturer's instructions (MyBioSource, San Diego, Calif., USA).

To test biocompatibility of IPN hydrogel, chondrogenic progenitor cells (CPCs) were isolated as described in Seol et al. (2012) and were encapsulated in IPN hydrogel disks (5 10⁶ cells/mL) for in vitro viability assay using LIVE/DEAD staining as described in Sukegawa et al. (2012) at different time point (Day 1, 7, 21).

### SDF-1α and its Receptor CXCR4 Expression

To assess SDF-1α and its receptor CXCR4 expression upon cartilage focal injury, immunofluorescence staining was used for cell surface markers using monoclonal anti-CXCL12 antibody (Abcam, Cambridge, Mass.) and anti-CXCR-4 antibody (Santa Cruz Biotechnology, Inc., Dallas, Tex., USA). A goat anti-mouse fluorescent secondary antibody (Alexafluor 488) was used for fluorescent labeling and detection (Jackson lmmunoresearch, West Grove, Pa.) using confocal microscopy. Staining was performed on monolayer cultured chondrogenic progenitor cells (CPCs), normal chondrocytes (NCs), as well as on cryosections of impacted articular cartilage, and non-impact fresh cartilage tissue as described in Alsalameh et al. (2004). CXCL12 and CXCR4 expression were also compared between CPCs and NCs by real time RT-PCR following a method described in Seol et al. (2011). Each real-time PCR experiment was done with at least three replicates, and target gene expression is presented as normalized values to β-ACTIN.

IPN scaffold implantation, cell migration, and in vitro chondrogenesis

Osteochondral explants (12 mm of diameter and 8-10 mm of thickness) were harvested from the bovine femur condyle (12-18 months of age, 9 animals in total). After two days preequilibrium culture, full thickness chondral defects (4 mm of diameter and about 2 mm of thickness) were created as described in Seol et al. (2012), and maintained in culture overnight before IPN implantation. IPN (about 60 μL) with or without rhSDF-1α (100 ng/mL or 200 ng/mL) was implanted into defects slightly over the surface of the explants, which were then placed back to culture. To monitor cell migration, confocal microscopy was performed essentially as described in Seol et al. (2014). Cell numbers were quantified by averaging automated cell counts from 6 random 20× images using ImageJ. DNA content in IPN hydrogel was quantified following procedures in Seol et al. (2014). Empty IPN gel from same culture condition was used as blank control.

Upon cell migration by day 12, explants were incubated in chondrogenic medium (DMEM containing 10 ng/mL TGF-β1, 100 ng/mL IGF-1, 0.1 μM dexamethasone, 25 μg/mL L-ascorbate. 100 μg/mL pyruvate, 50 mg/mL ITS+ Premix and antibiotics) at 5% CO₂, 37° C. for up to 6 weeks. Regenerated tissue together with host cartilage were harvested from explants and analyzed for extra cellular matrix formation using Safranin-O/fast green staining of either cryosections (3 weeks) or paraffin-fixed sections (6 weeks).

### Immunohistochemical, Biochemical and Ultrastructural Evaluation of Cartilage Repair

For immunohistochemistry analysis, deparaffinized sections from samples of 6 weeks were stained with type II collagen and aggrecan antibodies (Developmental Studies Hybridoma Bank, Department of Biology, The University of Iowa, Iowa City, Iowa, USA). A goat anti-mouse secondary antibody (Vector Laboratories, Inc., Burlingame, Calif.) was used for detection. The reaction products were visualized by Vectastain ABC kit and the DAB Peroxidase Substrate Kit (Vector laboratories, Inc., Burlingame, Calif., USA), according to the manufacturer's instructions. Lubricin, an articular cartilage superficial zone protein, staining was also performed using a Rabbit polyclonal antibody, and detected with a goat anti-rabbit secondary antibody (Vector Laboratories, Inc., Burlingame, Calif., USA). All negative controls were performed using same staining without using primary antibodies. Dimethyl methylene blue (DMMB) dye-binding assay was used for quantifying sulfated glycosaminoglycan (sGAG) content as previously described.

The water content between cartilage repair tissue and native cartilage was compared, while blank IPN hydrogel was used as negative control. All samples were measured for their wet weight with a bench top scale (Mettler-Toledo, LLC, Columbus, Ohio, USA), as well as dry weight after lyophilization (Lobconco, Kansas City, Mo., USA) overnight at −45° C. Water content was determined by following calculation: water content=(wet weight−dry weight) i wet weight×100%. The cartilage tissues were harvested 6 weeks after chondrogenesis as well as freshly fabricated IPN gel. SEM samples were processed using methods in Swords et al. (2002), and all scanning electron microscopy was performed at the University Of Iowa Central Microscopy Research Facility (CMRF).

### Biomechanical Assessment of Tissue Repair and Material Properties of Regenerated Tissue

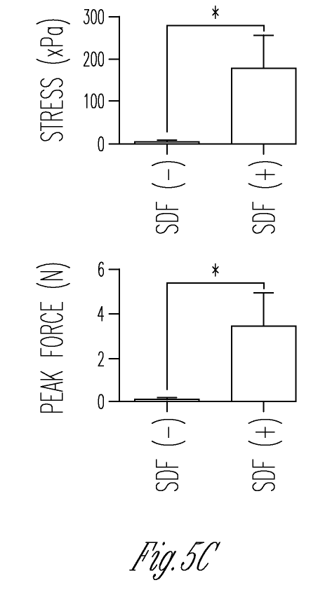

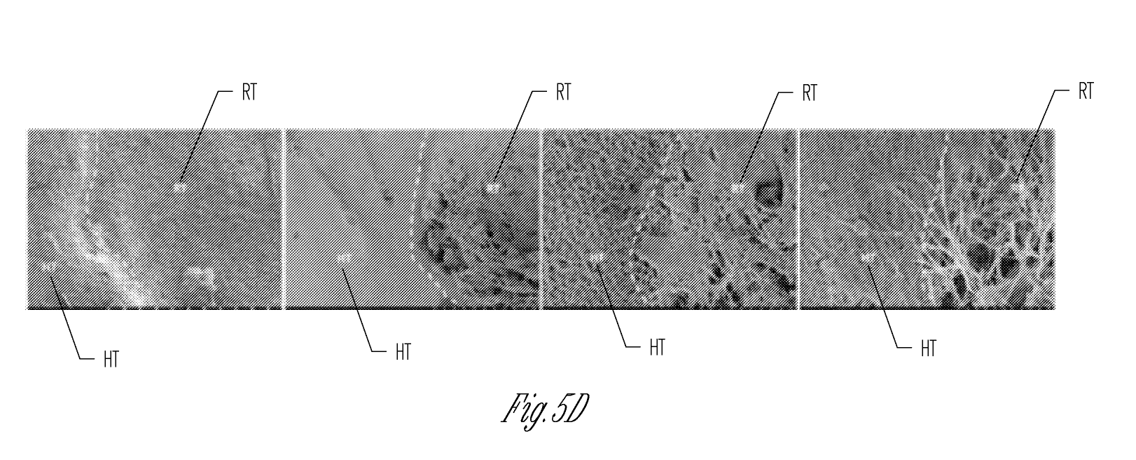

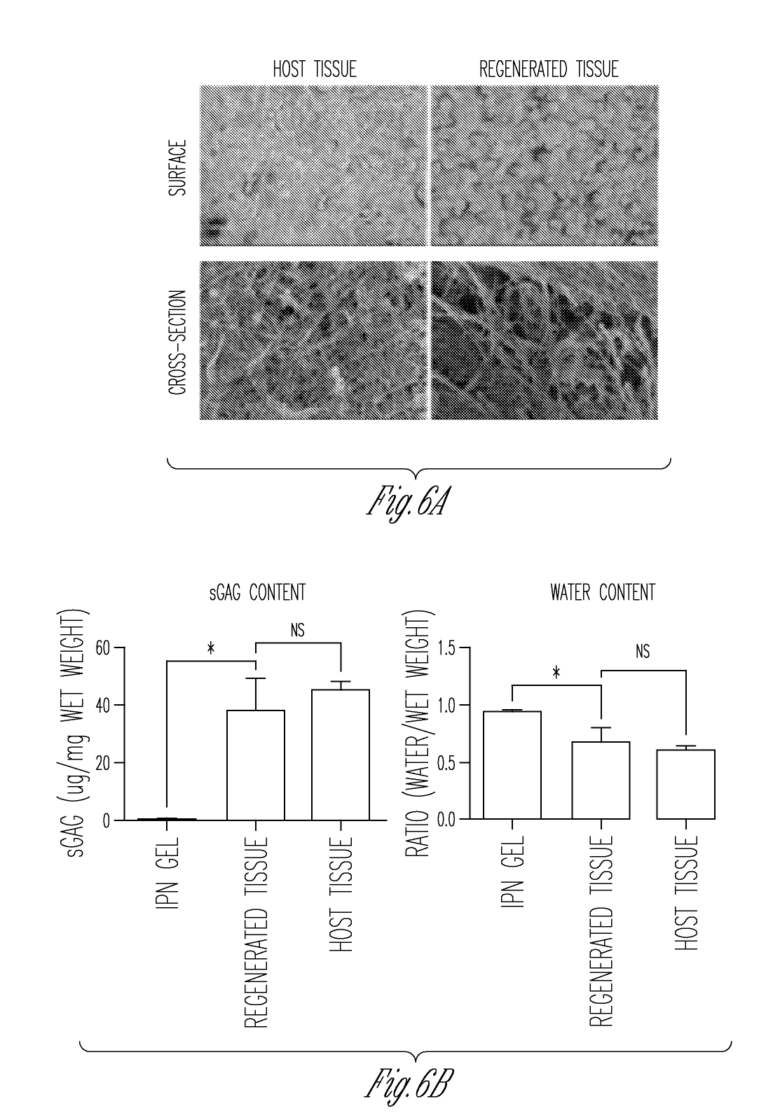

In order to evaluate integration strength between repair and host cartilage tissue, a “push-out” test was performed for both SDF treated groups (n=9) and non-treated groups (n=6). A customized cartilage fixation device rigidly held samples to measure integration (FIG. 5B). Upon harvesting, the specimens were then placed in the fixation device while a LabVIEW (National Instruments Corporation, Austin, Tex., USA) controlled stepper motor (Ultra motion, Cutchogue, N.Y., USA) depressed a cylindrical indenter (3.8 mm diameter) connected to a load cell (1 Kg Honeywell, 1 KHz sample rate) at a constant velocity of 0.1 mm/s (FIG. 5B, dashed inset). The test proceeded through the full thickness of the tissue, and the integration strength was determined by maximum force recorded divided by the area of integration.

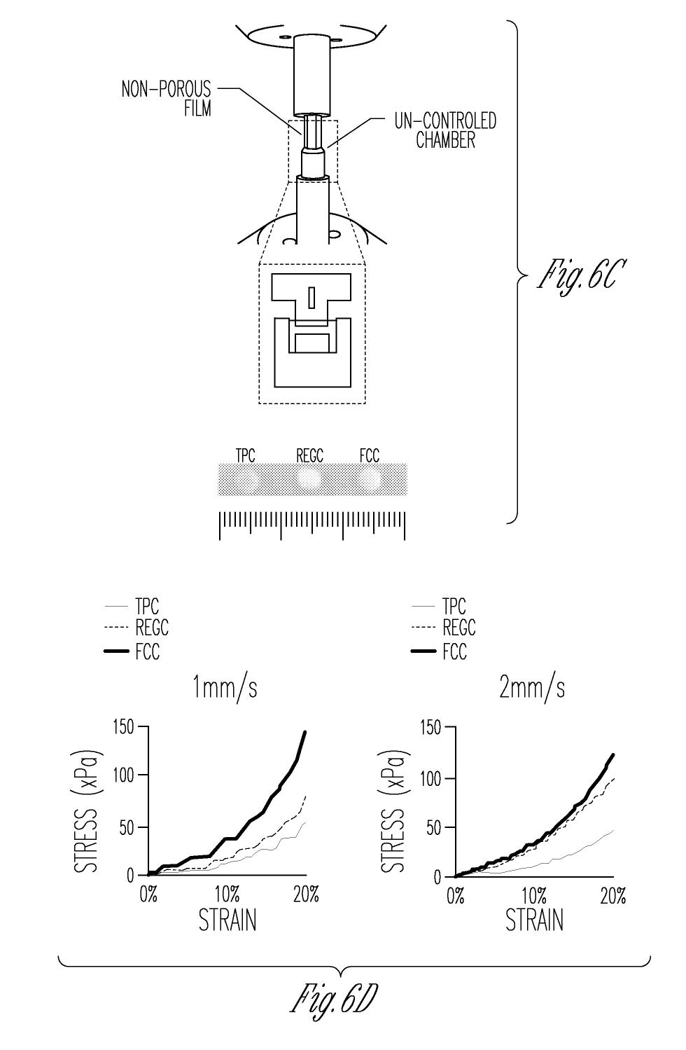

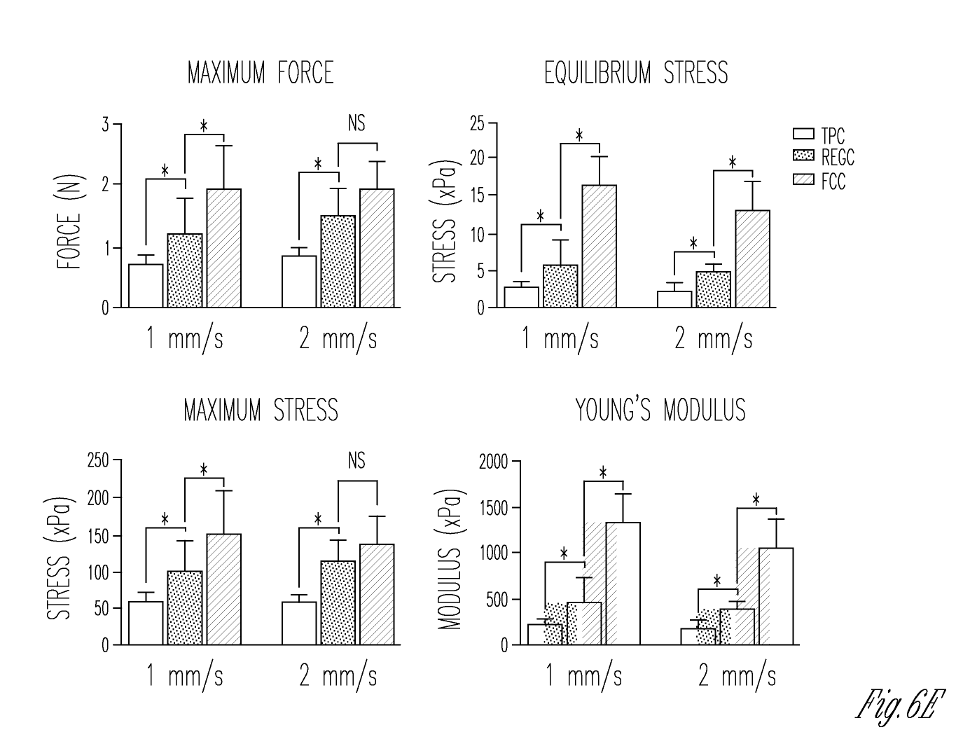

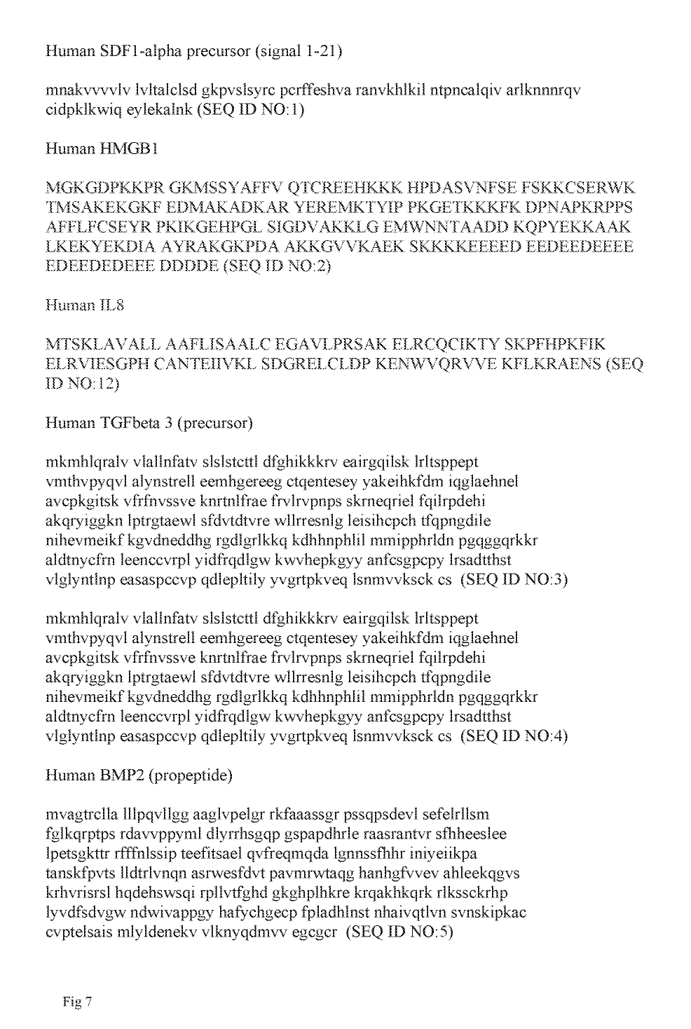

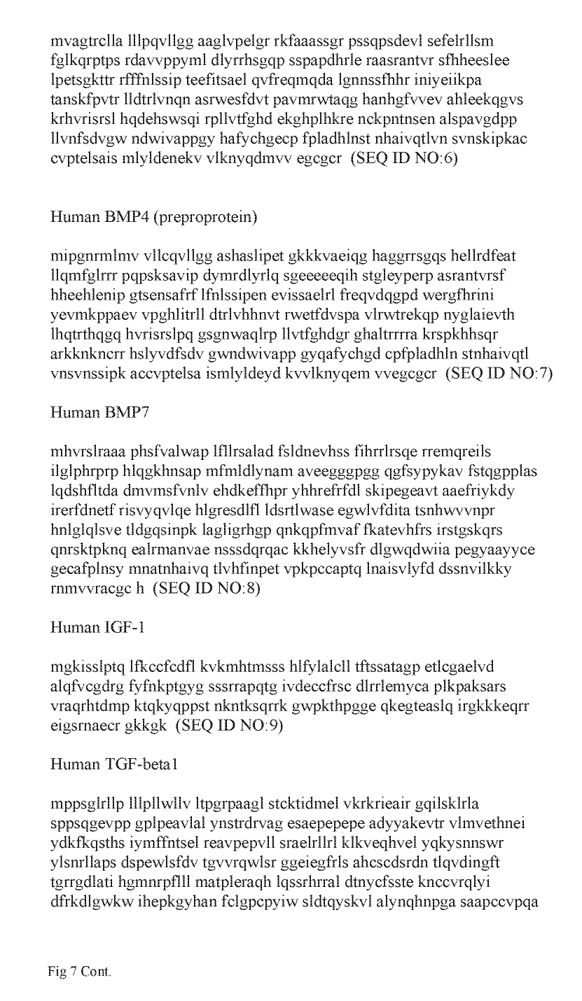

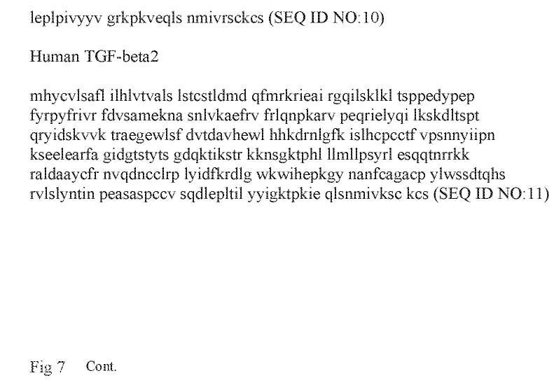

To further characterize the mechanical property of regenerated cartilage tissue, stress relaxation tests were performed on regenerated cartilage as well as native cartilage tissue harvested from the explants using a MTS Insight materials testing machine (MTS Systems Corporation, Eden Prairie, Minn., USA) (FIG. 6C top). Briefly, cartilage samples' thickness was measured by a laser measurement system (KEYENCE CORPORATION OF AMERICA, Itasca, Ill., USA) and placed in an unconfined chamber. A non-porous platen was brought into contact with the tissue surface and the tissue was compressed to 20% strain at 1 mm/s or 2 mm/s velocity. A 10 N load cell recorded the load as compression to 20% strain was held for 20 minutes. Maximum stress, equilibrium stress, Young's modulus, and maximum force were recorded or calculated. This test was applied to regenerated cartilage (REGC, n=9) formed in defects filled with IPN contained SDF-1α and were cultured for 6 weeks. Native cartilage samples were harvested from tibial plateau (TPC, n=8) or femur condyle (FCC, n=8) of healthy bovine knee joint, respectively (FIG. 6C bottom).

### Statistical Analysis

All data are presented as the mean SD and were analyzed by GraphPad Prism 6 (GraphPad Software, Inc., La Jolla, Calif., USA) using Student's t-test. P values less than 0.05 were considered significant.

### Results

### Fabrication and Characterization of IPN Ccaffold

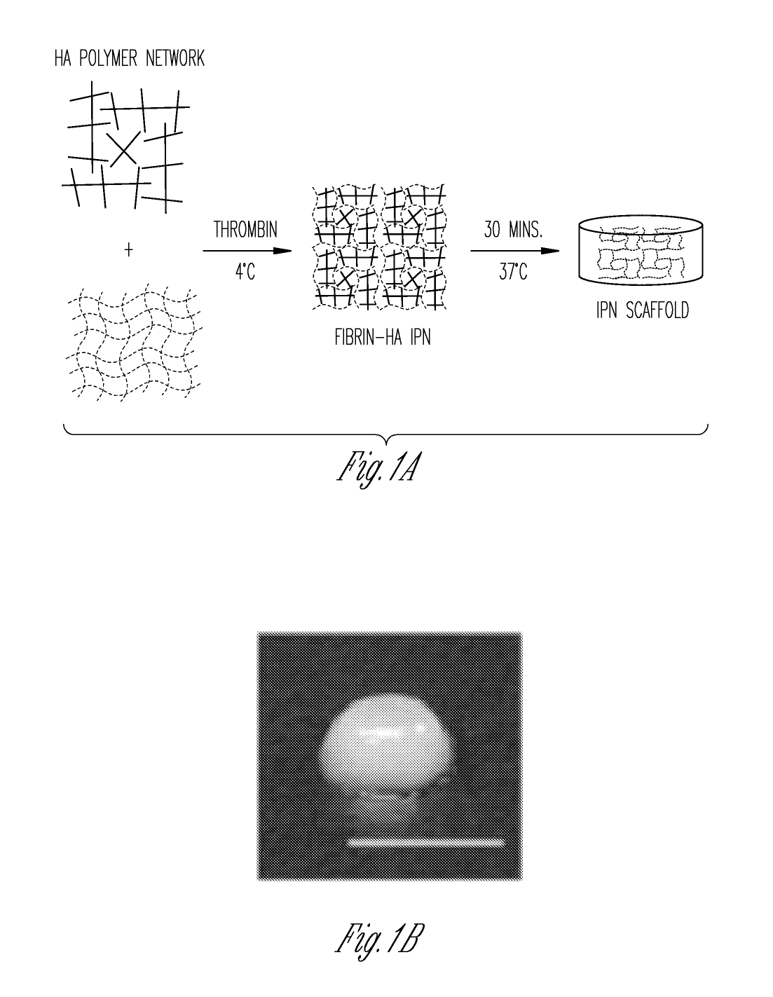

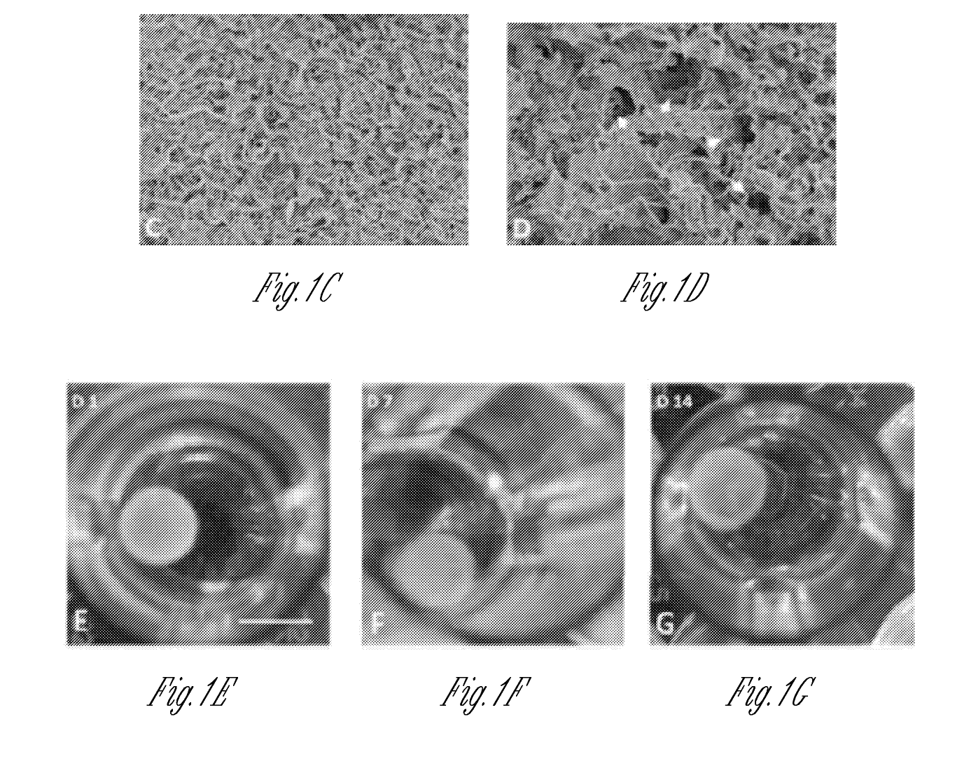

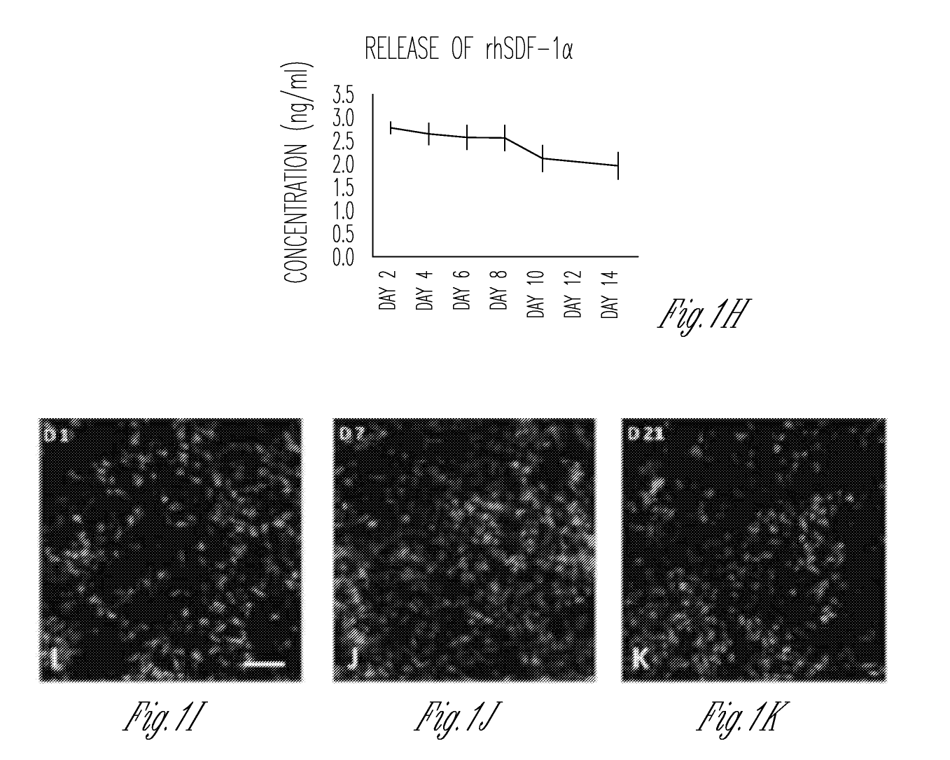

IPN hydrogel can be readily formed by thrombin initiated cross-linking of fibrinogen to become fibrin fibers, and fully polymerized with defined shape under physiological temperature (37° C.) with HA network fully penetrated the pores among fibrin fibers (FIG. 1A). After polymerization, the IPN scaffold displayed an opaque appearance, and a well-defined disk shape (FIG. 1B). SEM images showed HA network was fully distributed within fibrin fibers with great homogeneity and interconnected pore (arrow heads), both from the surface (FIG. 1C) and the cross-section (FIG. 1D). This porous structure would allow cells to attach and migrate both along the surface, and within implanted IPN scaffold.

IPN scaffold maintained its integrity in PBS as long as 2 weeks without noticeable changes (FIG. 1E-G). The time-dependent release curve showed that rhSDF-1α could be released over 14 days (FIG. 1H), with daily protein concentration maintained at over 2.0 ng/mL, and still with a continuous releasing trend. CPCs were encapsulated in IPN scaffold. To check their biocompatibility in terms of cell viability, confocal imaging was employed. Confocal images showed minimal number of dead cells (red fluorescence), while most of the cells were viable (green fluorescence) (FIG. 1I-K). The initial encapsulation process yielded a cell viability of 91.6±2.4 at day 1, and cell viability continued to maintain in high level (≧90%) at day 7 and day 21, respectively (FIG. 1L). These data suggested IPN scaffolds are easy to fabricate, able to support sustained release of rhSDF-1α, and biocompatible

SDF-1α/CXCR4 Expression and rhSDF-1α Guided CPCs Migration.

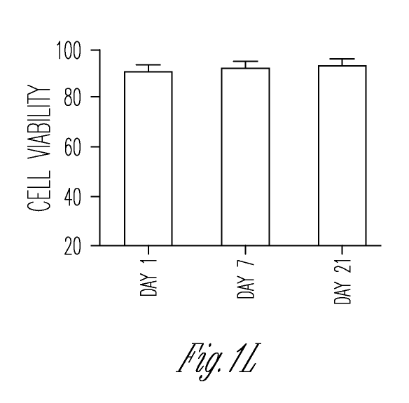

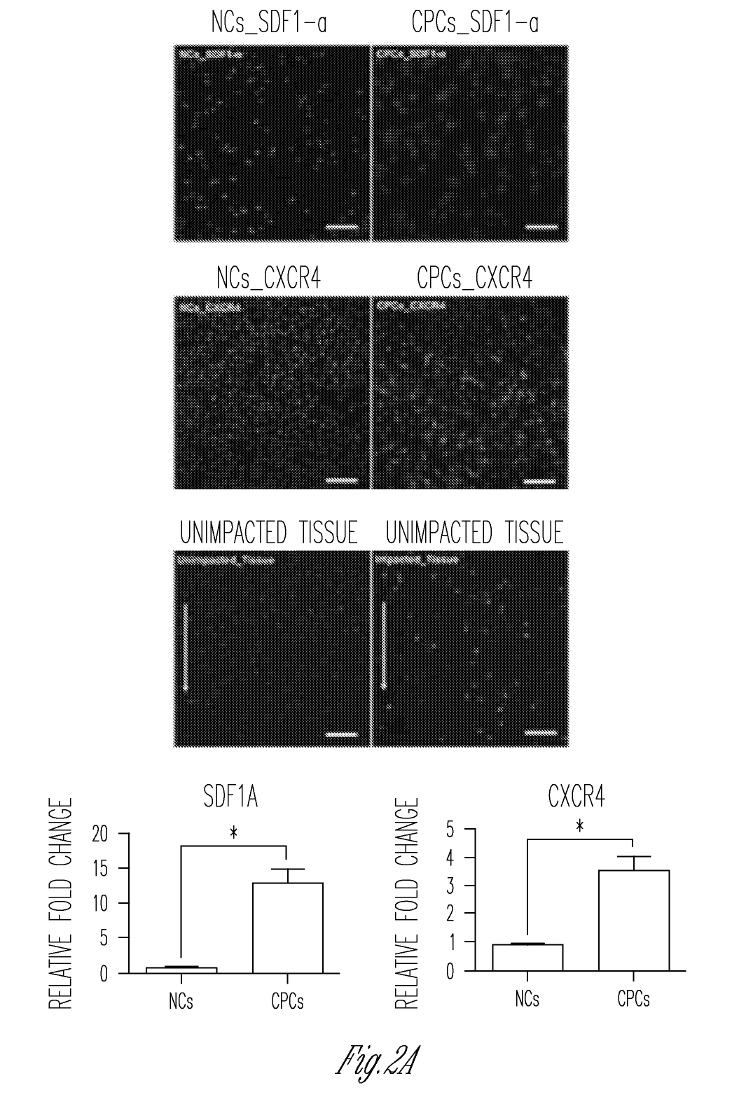

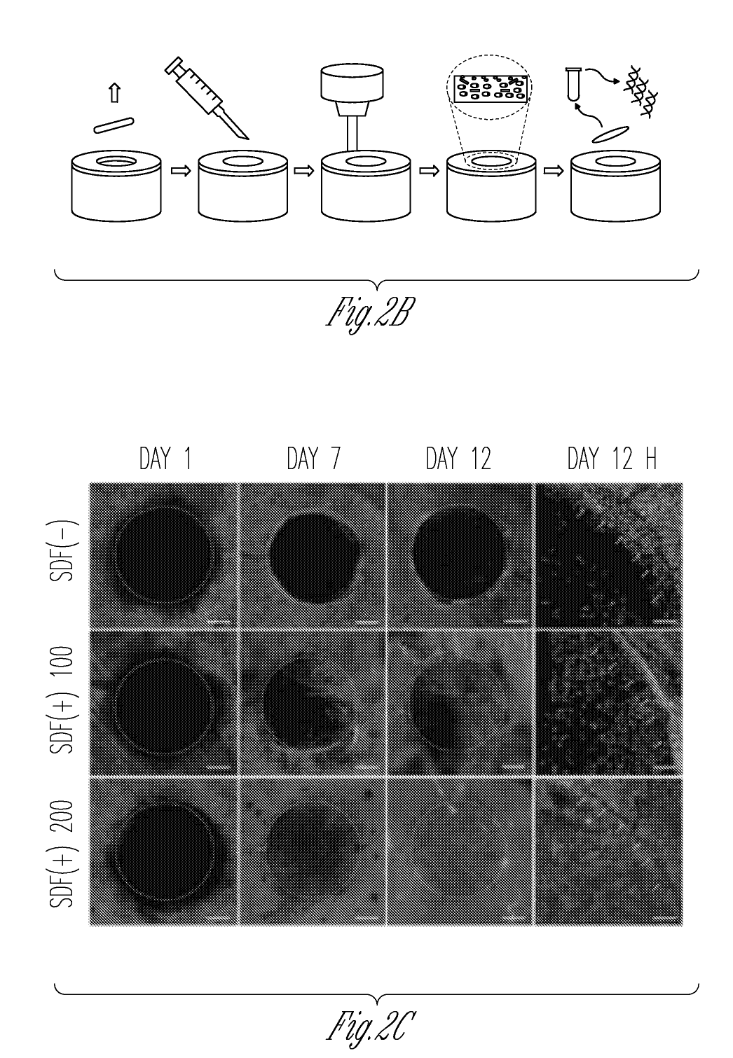

Immunofluorescence staining showed high expression of SDF-1α protein in CPCs with over 90% cells positively stained (FIG. 2A, upper right). In contrast, the SDF-1α protein expression was barely detectable in NCs (FIG. 2A, upper left). A similar pattern was observed for CXCR4 as well (FIG. 2A, middle). For impacted cartilage, SDF-1α also showed significantly increased expression (FIG. 2A, lower right) compared with non-injured freshly isolated cartilage (FIG. 2A, lower left) throughout the full depth of the tissue, with stronger expression on the superficial/middle zone (arrow pointing from superficial to deep zone). For RT-PCR, SDF-1α and CXCR4 mRNA expression was 13-fold and 3.5-fold higher in the CPCs compared with NCs, respectively (P=0.0004).

Upon creation of full-thickness articular cartilage defect and implantation of IPN in the absence (PBS) or presence of rhSDF-1α (100 ng/ml and 200 ng/ml), cell migration was monitored at different time points by confocal microscopy (FIG. 2B). As clearly shown in FIG. 2C, in explants implanted with rhSDF-1α free IPN, very few cells migrated into the defect area over 12 days, and the migrated cells were mainly at the defect edge, leaving the majority of the defect empty. For explants implanted with rhSDF-1α loaded IPN, significant number of cells migrated from the peripheral area to the center of the defect at day 7 and more cells at day 12. Cell migration also displayed an rhSDF-1α concentration dependent manner, with increased numbers of migrating cells at the higher dose (200 ng/mL) of rhSDF-1α either at day 7 or day 12, Thus, 200 ng/rnL rhSDF-1α was used in additional studies for full-thickness cartilage repair.

To further quantify the effect of rhSDF-1α on progenitor cells migration, high magnification confocal images from Day 12 (Day 12H) were used for automated cell counting. IPN loaded with rhSDF-1α (200 ng/mL) attracted over 250% (P<0.0001) as many cells as that in IPN scaffold without rhSDF-1α, Similarly, ds DNA content on day 12 was over 2-fold increase in rhSDF-1α (200 ng/mL) loaded IPN compared with rhSDF-1α free IPN (FIG. 2D), while not significantly higher than rhSDF-1α (100 ng/mL) group. These observations suggest that exogenous rhSDF-1α could act as a chemotactic cue for initiation of progenitor cell homing to repopulate full-thickness cartilage defect filled with IPN.

### Histology and Immunohistochemistry of Repaired Cartilage Tissue

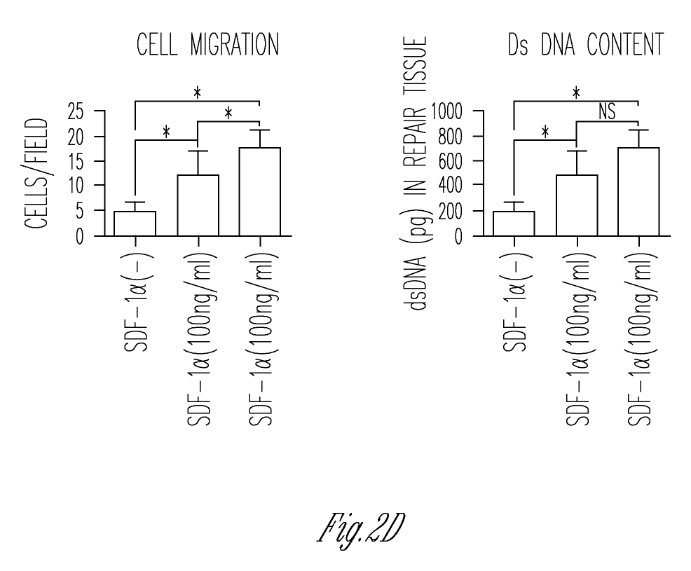

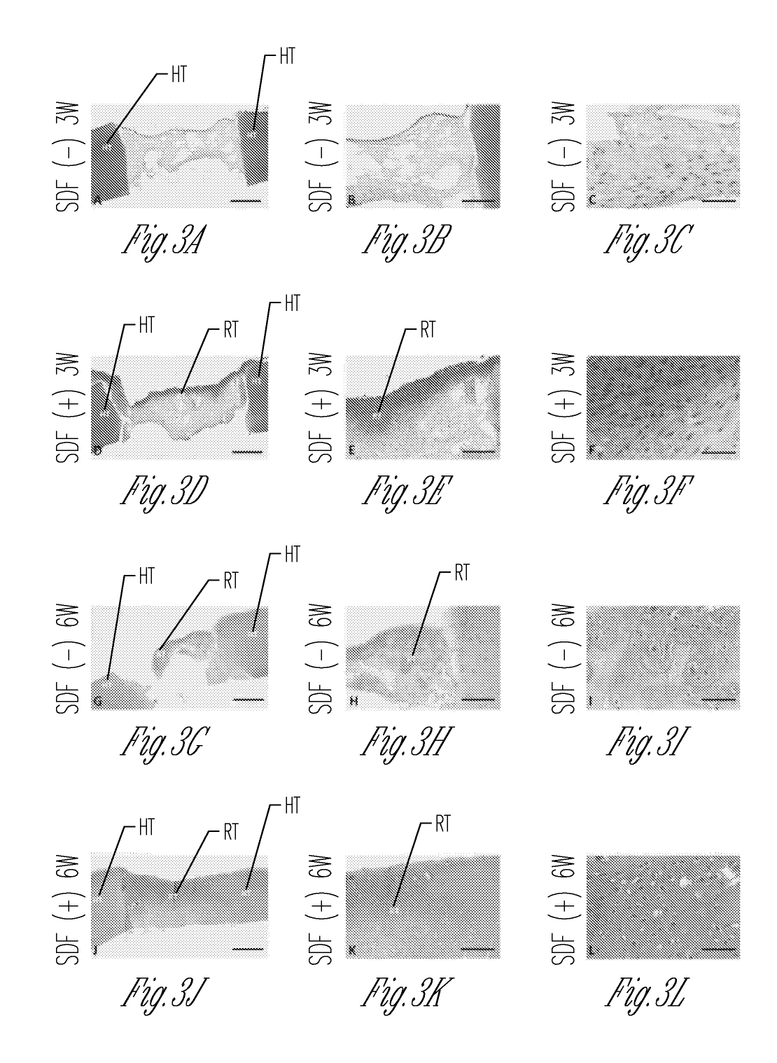

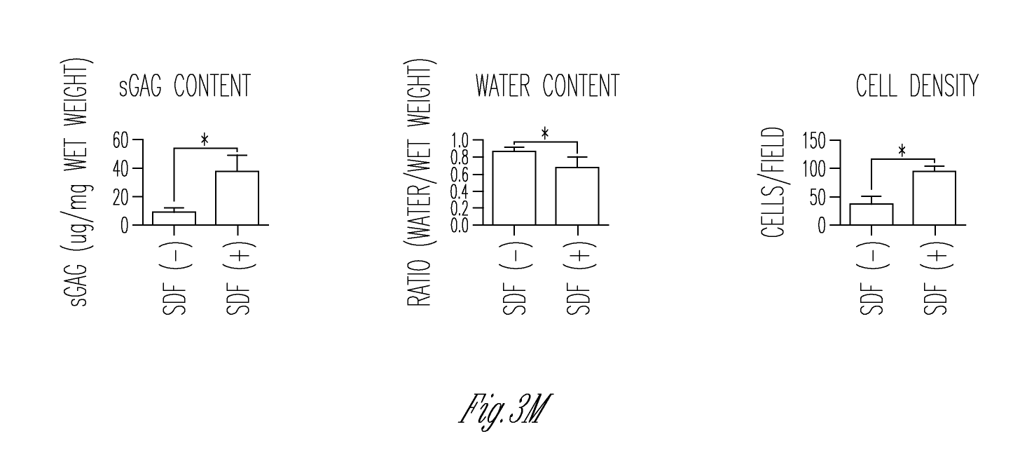

Histological evaluation of repaired cartilage defect was carried out at the end of 3 weeks and 6 weeks for cartilage ECM production. 3 weeks after chondrogenic induction, substantially higher amount of proteoglycan deposition was observed in rhSDF-1α loaded IPN scaffold with strong positive staining for Safranin-O (FIG. 3D) compared with IPN only scaffold, which mainly displayed fast-green staining only (FIG. 3A). Stronger Safranin-O staining was observed on the superficial zone of regenerated cartilaginous tissue and gradually decreased to the deep zone (FIG. 3E). Most of the migrated cells still displayed a spindle-like morphology (FIG. 3C&F), more like that of CPCs (Seol et al., 2012), 6 weeks after chondrogenic differentiation, both IPN only scaffold and rhSDF-1α loaded IPN scaffold showed increased proteoglycan deposition and stronger staining for Safranin-O (FIG. G&J) compared with those at 3 weeks. The rhSDF-1α loaded IPN scaffold yielded evenly distributed cells and more intense Safranin-O positive staining for both pericellular and inter-territorial extra cellular matrix (ECM) nearly throughout whole depth of regenerated tissue (FIG. 3K). In contrast, rhSDF-1α free IPN scaffold had disorganized cell distribution and newly synthesized proteoglycan with positive but moderate Safranin-O staining mainly for pericellular ECM (FIG. 3H). Characteristic cobble stone-like morphology was observed for migrated cells as a sign of complete differentiation into chondrocytes (FIG. 3I&L), with cells in the rhSDF-1α loaded IPN scaffold having more similarity to host chondrocytes (FIG. 3L).

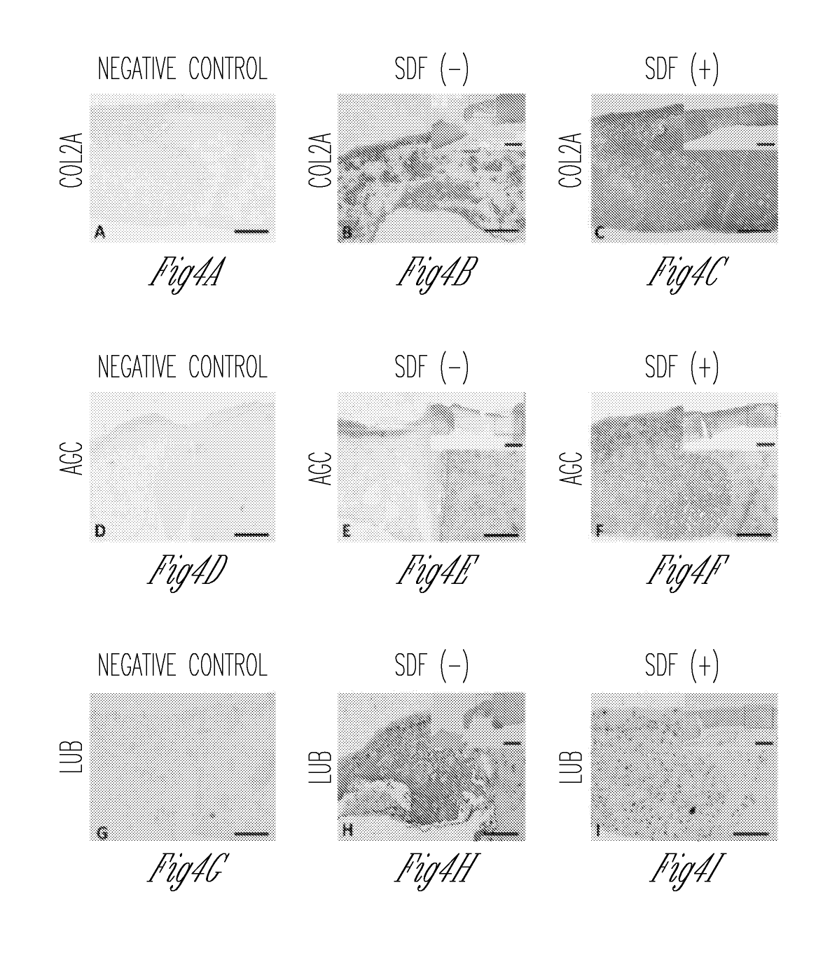

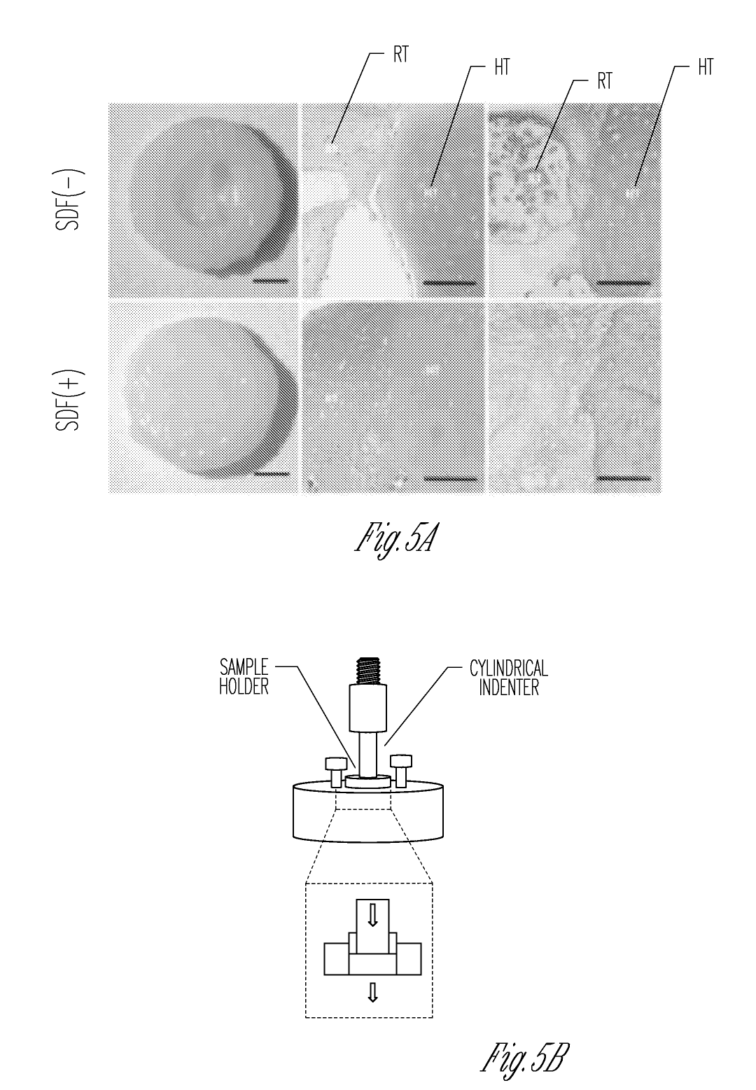

Further quantification of sulfated glycosaminoglycan (sGAG) by DMMB assay showed that rhSDF-1α loaded IPN scaffold yielded nearly 8-fold (P=0.0055) higher sGAG content than rhSDF-1α free IPN scaffold (FIG. 3M left). Moreover, regenerated cartilage tissue from rhSDF-1α loaded IPN scaffold had significantly ((P=0.0242)) lower water content than that from rhSDF-1α free IPN scaffold (FIG. 3M middle). Quantification of cell density for each high magnitude histology image showed over twice (P<0.0001) as many cells in IPN+rhSDF-1α group as that in IPN only group (FIG. 3M right). Interestingly, higher cell density was observed in cartilage repair tissue compared with native cartilage from histology images (FIGS. 3D and 4J), and cell density in repair tissue gradually decreased from superficial/middle zone to deep zone. This may explained in that most of the CPCs were from articular cartilage superficial zone, and the migrated CPCs were highly proliferative (Seol et al., 2012).

Immunohistochemistry showed massive type II collagen as well as aggrecan positive staining throughout the repair tissue from rhSDF-1α loaded IPN, nearly identical from native cartilage tissue (FIG. 4C&F). In contrast, repair tissue from rhSDF-1α free IPN displayed uneven and isolated areas of collagen type II and aggrecan staining, mainly pericellular and on the superficial zone, leaving the majority of ECM lack of positive staining (FIG. 4B&E). rhSDF-1α loaded IPN scaffold yielded regenerated tissue with strong positive staining of lubricin on the superficial zone, while relatively fewer positively stained cells in the middle and deep zone, largely similar to that in native cartilage (FIG. 4I). However, repair tissue from SDF free IPN only had disordered lubricin staining cluttered within ECM (FIG. 4H). A great continuity of all three type of staining across the surface of native tissue and repair tissue was also observed in rhSDF-1α loaded IPN (insets of FIGS. 4C, F, and I), indicating possible potential of restoring defected articular cartilage surface. All negative controls were only lightly stained for the background (FIGS. 4A, D, and G).

Integration of Repair Tissue with Native Cartilage

Integration with native tissue is a milestone of successful repair. A great deal of repair and host cartilage tissue connection both macroscopically, histologically and ultrastructually, was observerd. The defect from SDF (+) group showed nearly seamless repair and integration with host cartilage, while defect from SDF (−) group lacks tissue regeneration with evident defect remaining unrepaired. Similarly, both histology and immunostaining images showed significantly improved repair-host tissue connection upon rhSDF-1α treatment with subsequent chondrogenesis (FIG. 5A).

The push-out test showed dramatically different integration strength between SDF (+) and SDF (−) groups. Both stress and peak force was significantly higher in rhSDF-1α treated groups than in untreated control groups (158.0±26,04 kPa vs. 7.56±1.34 kPa; 3.23±0.53 N vs. 0.15±0.03 N, respectively) (FIG. 5C). In addition, SEM images showed integration of regenerated tissue with host cartilage both for cell ingrowth and ECM fibers cross-linking. The defect line was largely closed by interconnected ECM fibers from both native and regenerated tissue in SDF (+) group (FIG. 5D).

### Biochemical and Mechanical Properties of Regenerated Cartilage Tissue

The ultrastructure of regenerated tissue and native cartilage tissue, as well as their sGAG content, water content and various material properties, were compared. From SEM images, cells in regenerated tissue displayed a slightly isolated form from their surrounding ECM, while cells in host cartilage were well resided in the ECM with tight attachment to their lacunae. Also, cell density was relatively higher in regenerated tissue in comparison to native tissue (FIG. 6A, upper panel). Collagen fibers formed a less compacted network in regenerated cartilage compared with native cartilage (FIG. 6A, lower panel), which may result in differential mechanical properties of two cartilage tissues. DMMB assay showed that sGAG content significantly (P=0.0016) increased in regenerated tissue in regard to control IPN scaffold, while not significantly different (P=0.2607) from host cartilage tissue. Similarly, significantly (P=0.0016) decreased water content presented in regenerated tissue compared with control IPN scaffold, but no significant differences existed between host cartilage and regenerated cartilage.

For mechanical properties, regenerated cartilage (REGC) generally had all four measurements (maximum and equilibrium stress, Young's modulus, and maximum force) higher than tibial plateau cartilage (TPC), while lower than femur condyle cartilage (FCC) at two testing speed. Empty IPN gel was not measurable under current testing system due to its low mechanical property. REGC presented a Young's modulus of 746.7±82.3 kPa (1 mm/s) and 965.4±78.9 (2 mm/s), which are notably higher than that of TPC (475.6 ±42.9 at 1 mm/s and 542.8±46.1 at 2 mm/s, respectively). Young's modulus of REGC reached nearly 70% of that of FCC at both testing speeds. Similarly, other properties of REGC were all within the physiological range (TPC to FCC) of native bovine cartilage. Notably, REGC showed an increased Young's modulus with higher loading speed, similar to TPC and FCC.

### Discussion

The development of novel cartilage repair strategies by stimulating endogenous cell homing is of substantial clinical interest. In this study, for the first time it was demonstrated that full thickness cartilage defects could be repaired entirely by endogenous progenitor cells from articular cartilage, rather than from multiple sources like in other studies (Sukegawa et al., 2012; Zhang et al,, 2013; Mendelson et al., 2011), demonstrating the intrinsic cartilage healing potential can be enhanced by a two-step strategy to first initiate progenitor cell chemotaxis with rhSDF-1α, followed by stimulation of chondrogenesis.

The expression of SDF-1α and CXCR4 upon cartilage injury supports the involvement of the SDF-1/CXCR4 axis in migration of CPCs to the site of cartilage defect, SDF-1α also significantly increased progenitor cell migration from surrounding cartilage into IPN scaffolds, clearly demonstrating its ability to direct progenitor cells homing. These results are consistent with a number of published studies (Shen et al., 2014; Thevenot et al., 2010; Schantz et al., 2007; Shen et al., 2010; Kitaori et al., 2009). Subsequent chondrogenic induction further stimulated type II collagen and aggrecan deposition, resulting in proteoglycan-rich cartilage matrix. A more zonally organized lubricin staining may suggest the potential for regenerating stratified articular cartilage with zone specific properties. Comparison between regenerated tissues by rhSDF-1α loaded IPN and native cartilage showed great similarities, in terms of sGAG content, water content as well as ultrastructural collagen fiber alignment and cell-ECM interaction, which are all essential elements to establish articular cartilage function.

Integration strength determines the bonding of engineered cartilage with surrounding native tissue (Obradovic et al., 2001). The present study showed dramatically higher integration strength by using rhSDF-1α loaded scaffold, which was up to 158.0±26.04 kPa, more than three times higher than that reported in comparable studies (Diekman et al., 2012; Tam et al., 2007; Theodoropoulus et al,, 2011). This may indicate that more migrated CPCs would contribute to enhanced tissue integration. In fact, Lu et al. demonstrated that more migrated chondrocytes at the interface of engineered cartilage and surrounding cartilage could result in dramatically stronger integration after autologous chondrocyte implantation (Lu et al., 2013). It is also worth noting that the collagen fiber networks of the regenerated and host tissues in the fully treated defects were extensively entangled with each other, which might explain the gain in integration strength as well.

Regeneration of mechanically functional cartilage tissue is one of the measures of success of any cartilage repair strategy. Although engineering cartilage with primary chondrocytes has reached physiological equivalence with native cartilage for compressive moduli, only no more than 50% was achieved for cartilage engineered from stem/progenitor cells to date. In our study, large full thickness chondral defect were successfully repaired in vitro by cartilage tissue with Youngs modulus in the physiological range in relatively short time. Further improvement of may require mechanical loading stimulation, which has been shown to enhance Young's modulus of tissue engineered cartilage (Bian et al., 2010). For in vivo translation, certain immobilization procedures may be needed for the IPN gel during the early stages of neocartilage development, after which physiological loading would be beneficial for its further maturation.

In the future, more strategies could be developed to not only incorporating chemotactic factors for cell homing, but to modify scaffolds by introducing anti-inflammatory agents, which would certainly have profound benefits for cartilage neo-genesis. All chemokines, growth factors (Eswaramoorthy et al., 2012) or genetic materials (Ha et al,, 2012), or other agents, can be encapsulated within polymer microspheres to achieve sustained or multi-phase release from the scaffold into the joint defects.

### CONCLUSION

A cartilage repair strategy was developed that exploits the regenerative potential of endogenous chondrogenic progenitor cells. The matrix formed by these cells is similar in composition to native cartilage and strongly adheres to surrounding tissues. Regenerated cartilage tissue possesses mechanical properties within the physiological range for functional native cartilage. Optimization of this strategy could lead to a minimally invasive, single-step procedure for cartilage repair.

### REFERENCES

Alsalameh et al., Arthritis and Rheum., 50:1522 (2004).

Bian et al., Tissue Engineering Part A, 16:1781 (2010).

Dealy, Rheumatology: Curr. Res. S3:e001 (2012).

De Bari et al., Arthritis Rheum, 44:1928 (2001).

Diekman et al., Proc. Nat. Acad. Sci., 109:19172 (2012).

Dowthwaite et al., J. Cell sci., 117:889 (2004).

Erickson et al., Acta Biomat., 8:3027 (2012).

Eswaramoorthy et al., Acta Biomaterialia, 8:2254 (2012)

Fodor, Reprod. Biol. Endocrin., 1:102 (2003).

Ha et al., Cytotherapy, 14:247 (2012).

Hattori et al., Blood, 97:3354 (2001).

Johnstone et al., Eur. Cells Mater., 25:248 (2013).

Joos et al., Arthritis Res. & Ther., 15:R119 (2013).

Kitaori et al., Arthritis and Rheumatism. 60:813 (2009).

Klein et al., Macromol. Bio., 9:1049 (2009).

Koelling et al., Cell Stem Cell, 4:324 (2009).

Lagasse et al., Nature Med., 6:1229 (2000).

Lee et al., Lancet, 376:440 (2010).

Lu et al., Tissue engineering Part A, 19:2506 (2013).

Mendelson et al., J. FASEB, 25:3496 (2011).

Obradovic et al., Orthopaedic Res. Soc., 19:1089 (2001).

Pittenger et al., Science, 284:143 (1999).

Prockop, J, Am. Soc. Gene Ther., 17:939 (2009),

Rarnpichova et al., ASAIO J., 56:563 (2010).

Schantz et al., Tissue Eng. 13:2615 (2007).

Seol et al., Arthritis and Rheumatism, 64:3626 (2012),

Seol et al., BMC Res., 4:162 (2011).

Seol et al., Tissue Eng. Part A, 20:1807 (2014).

Shen et al., Biomaterials, 31:7239 (2010).

Shen et al., Stem Cells Transl. Med., 3:387 (2014).

Slaughter et al., Adv. Mater., 21:3307 (2009).

Sukegawa et al., Tissue Eng. Part A, 18:934 (2012).

Swords et al., Infect. and Immun., 70:4661 (2002).

Tam et al., Orthopaedic Research Society, 25:1136 (2007).

Theodoropoulos et al., Clinical Orthopaedics and Related Res., 469:2785 (2011).

Thevenot et al., Biomaterials, 31:3997 (2010).

Tuan, Arthritis Rheum., 54:3075 (2006).

Wickham et al., Clin. Orthop. Rel. Res., 412:196 (2003).

Yu et al., J. Biomech. Eng., 135:91011 (2013).

Zhang et al., Biomaterials, 34:713 (2013).

### Abbreviations, Acronyms and Symbols

OA: Osteoarthritis
CPCs: Chondrogenic progenitor cells
NCs: Normal chondrocytes
rhSDF-1α: recombinant human stromal cell-derived factor 1 alpha
BMSCs: Bone marrow mesenchymal stem cells
ASCs: Adipose stem cells
CXCR4: chemokine (C-X-C motif) receptor 4
HA: Hyaluronic acid
IPN: Interpenetrating polymer network
ECM: Extra cellular matrix
DPBS: Dulbecco's phosphate-buffered saline
ELISA: Enzyme-linked immunosorbent assay
RT-PCR: Reverse transcription polymerase chain reaction
DMEM: Dulbecco's Modified Eagle Medium
DNA: Deoxyribonucleic Acid
TGF-β1: Transforming growth factor beta 1
IGF-1: Insulin-like growth factor 1
DMMB: Dimethylmethylene
sGAG: Sulphated glycosaminoglycans
SEM: Scanning electron microscopy
REGC: Regenerated cartilage
TPC: Tibial plateau cartilage
FCC: Femur condyle cartilage
mRNA: Messenger ribonucleic acid
COL2A: Type II collagen
AGC: Aggrecan
LUB: lubricin
iPSCs: Induced pluripotent stem cells
BMP: bone morphogenetic protein
TKA: Total knee arthroplasty
TNF-α: Tumor necrosis factor alpha
IL-1β: Interleukin 1 beta
NO: Nitric oxide
MMP: Matrix metalloprotease

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A pharmaceutical composition comprising at least one naturally occurring polymer comprising an effective amount of an isolated protein that is a chemoattractant for chondrogenic progenitor cells and an effective amount of an isolated protein that is a chondrogenic factor, or nucleic acid sequence that encodes a chondrogenic factor.

2. The composition of claim 1 wherein the isolated protein that is a chemoattractant comprises an alarmin, IL-8 or SDF1-alpha, or any combination thereof.

3. The composition of claim 2 wherein the isolated protein that is a chemoattractant has at least 80% amino acid sequence identity to one of EQ ID NO: 1, 2 or 12.

4. The composition of any one of claims 1 to 3 wherein the natural occurring polymer comprises hyaluronic acid, fibrin, type I collagen, or a combination thereof.

5. The composition of any one of claims 1 to 4 wherein the chrondrogenic factor comprises a member of the TGF-beta superfamily.

6. The composition of claim 5 wherein the member has at least 80% amino acid sequence identify to one of SEQ ID Nos. 3-11.

7. The composition of claim 5 wherein the member comprises one or more of TGF-β1, -β2 or -β3, BMP2, BMP4, BMP7, or IGF-1.

8. The composition of any one of claims 1 to 7 wherein the isolated chondrogenic factor is encapsulated or complexed with nanoparticles or microparticles,

9. The composition of claim 8 wherein the nanoparticles or microparticles comprise lactic acid.

10. The composition of claim 8 wherein the microparticles or nanoparticles comprise poly(D,L-lactic-co-glycolic)acid (PLGA) or polyethyleneimine (PEI).

11. A method to enhance the repair of articular cartilage, to treat a joint injury or to prevent, inhibit or treat osteoarthritis in a mammal, comprising: administering to the mammal an effective amount of the composition of claim 1.

12. The method of claim 11 wherein the amount enhances the amount or level of normal hyaline cartilage.

13. The method of claim 11 or 12 wherein the composition is administered to a hip, knee or ankle of the mammal.

14. The method of any one of claims 11 to 13 wherein the composition comprises SDF1-alpha, HMGB1 or IL8, or any combination thereof.

15. The method of any one of claims 11 to 14 wherein the composition comprises nanoparticles or microparticles.

16. The method of claim 15 wherein the microparticles are formed of poly(D,L-lactic-co-glycolic)acid (PLGA) or polyethyleneimine (PEI).

17. The method of any one of claims 11 to 16 wherein the composition comprises TGF-β1, TGF-β2, TGF-β3, BMP2, BMP4, BMP7, or IGF1, or any combination thereof.

18. The method of claim 15 wherein the microparticles provide for sustained or extended release of the chondrogenic factor relative to the chemoattractant protein.

19. The method of any one of claims 11 to 18 wherein the mammal has a joint injury with at least one cartilage lesion.

20. The method of any one of claims 11 to 19 wherein the mammal as post-traumatic osteoarthritis.


SEQUENCE LISTING (12 sequences)

SEQ ID NO: 1 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

SEQ ID NO: 2 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 3 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 4 - protein, 412 aa, Homo sapiens; identical to SEQ ID NO: 3

SEQ ID NO: 5 - protein, 396 aa, Homo sapiens [sequence omitted]

SEQ ID NO: 6 - protein, 396 aa, Homo sapiens [sequence omitted]

SEQ ID NO: 7 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 8 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 9 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 10 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 11 - protein, 353 aa, Homo sapiens
MHYCVLSAFLILHLVTVALSLSTCSTLDMDQFMRKRIEAIRGQILSKLKLTSPPEDYPEPFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 12 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS